(19) United States
(12) Patent Application Publication
(10) Pub. No.: US 2012/0017905 A1
(43) Pub. Date: Jan. 26, 2012

(54) ARTIFICIAL NOSE AND BREATHING CIRCUIT PROVIDED WITH THE ARTIFICIAL NOSE

(76) Inventors: Yasuhiko Sata, Tokyo (JP); Norio Hachisu, Tokyo (JP)

(21) Appl. No.: 13/259,543

(22) PCT Filed: Mar. 11, 2010
(86) PCT No.: PCT/JP2010/054136

§ 371 Date: Sep. 23, 2011

(30) Foreign Application Priority Data
Apr. 6, 2009 (JP) 2009-092151

(51) Int. Cl. A61M 16/16

(52) U.S. Cl. 128/203.26

(57) ABSTRACT

Provided is an artificial nose and a breathing circuit provided with the artificial nose, including: an outer shell; a moisture permeable and water resistant film 6 disposed on an entire circumference of an internal surface of the outer shell, forming a water retention region with the outer shell, and forming an aeration region on an internal surface side thereof; a feed water inlet provided in the outer shell to supply water to the water retention region; a heat and moisture exchanger element loaded in the aeration region; and a heater disposed outside the outer shell, wherein the water supplied from the feed water inlet is retained in the water retention region by the moisture permeable and water resistant film, an inspiratory gas and an expiratory gas pass through the heat and moisture exchanger element loaded in the aeration region, and the artificial nose carries out a first heating and humidifying process of the inspiratory gas in which heat and moisture included in the expiratory gas passing therethrough are captured and retained by the heat and moisture exchanger element and the heat and the moisture are discharged to an inspiratory gas passing therethrough next, and a second heating and humidifying process in which only water vapor generated by heating of the heater passes through the moisture permeable and water resistant film and is supplied to the inspiratory gas passing through the heat and moisture exchanger element to heat and humidify the inspiratory gas and also the inspiratory gas in the heat and moisture exchanger element is heated by the heater.

### TECHNICAL FIELD

The present invention relates to an artificial nose heating and humidifying an inspiratory gas utilizing heat and moisture included in an expiratory gas of a person, and to a breathing circuit provided with the artificial nose.

### BACKGROUND ART

Among devices for artificial respiration and anesthesia and respiration of a person in whom a tracheostomy has been performed, an artificial nose (may also be referred to as an HME (Heat Moisture Exchanger)) is used as simple means of heating and humidifying an inspiratory gas that heats and humidifies an inspiratory gas utilizing heat and moisture is included in an expiratory gas of a person. Such an artificial nose is normally used at an end of a breathing circuit closest to a user, and is designed such that an inspiratory gas and an expiratory gas alternately pass through the artificial nose.

Here, as shown in FIG. 10, in a flow channel 112 of a conventional artificial nose 102, a heat and moisture exchanger element 114 is loaded that is configured with a foam having hygroscopicity, hygroscopic paper, or the like. The heat and moisture included in an expiratory gas exhaled from a user are captured and retained by the heat and moisture exchanger element 114, and the heat and moisture are discharged into an inspiratory gas that flows in the flow channel next to enable the heating and humidification of an inspiratory gas.

Here, the inspiratory gas heated and humidified sufficiently is generally considered to have a temperature of 37° C. and a relative humidity of 100%; and to realize this, 44 mg/L of moisture is required to be added to the inspiratory gas. Meanwhile, the maximum amount of water that can be transferred from the expiratory gas to the inspiratory gas by the heat and moisture exchanger element is approximately 30 mg/L, and thus sufficient moisture cannot be supplied to the inspiratory gas only by the heat and moisture exchanger element. In addition, since the heat of vaporization of water is large (for example, 586 cal/g at 20° C.), it is difficult to sufficiently vaporize water only by the heat included in the expiratory gas.

With that, to address this problem, a humidifier system is proposed that is provided with water supply means to refill moisture to a heat and moisture exchanger element and with a heater capable of heating the heat and moisture exchanger element (for example, refer to Patent Document 1).

### Prior Art Document

### Patent Document

Patent Document 1: Japanese Unexamined Patent Publication No. 2006-167447

### DISCLOSURE OF THE INVENTION

### Problems to be Solved by the Invention

In the humidifier system described in Patent Document 1, the water supply means is provided with a water permeable element (specifically, a hollow fiber bundle or a hollow fiber pipe) including a water permeable material, and the water filled in this water permeable element permeates a pipe wall to be supplied to the heat and moisture exchanger element. That is, the water supply means enables the moisture to be supplied to the heat and moisture exchanger element and the heater enables the heat to be supplied to the moisture exchanger element. Therefore, the water supply means and the heater can make up for the heat and the moisture that used to be insufficient in the heating and humidification of the inspiratory gas using the conventional heat and moisture exchanger element.

However, the pipe wall has water permeability in this humidifier system, so that there is a possibility of supplying excessive water to the heat and moisture exchanger element, and in this case, there are risks of obstructing the a flow channel of the inspiratory gas and the expiratory gas and of flowing water into a trachea or a lung of a user.

Accordingly, it is an object of the present invention to provide an artificial nose that solves the problems mentioned above, that is capable of humidification and heating of an inspiratory gas sufficient for a user in a safe state without risks of obstructing a flow channel of an inspiratory gas and an expiratory gas and flowing water into a trachea or a lung of a user, and also that is less affected by an external air or the like, and to provide a breathing circuit provided with the artificial nose.

### Means for Solving the Problems

To solve the problems mentioned above, one embodiment of an artificial nose of the present invention used for a breathing circuit is an artificial nose, includes: an outer shell; a moisture permeable and water resistant film disposed on an entire circumference of an internal surface of the outer shell, forming a water retention region with the outer shell, and forming an aeration region on an internal surface side thereof; a feed water inlet provided in the outer shell to supply water to the water retention region; a heat and moisture exchanger element loaded in the aeration region; and a heater disposed outside the outer shell, wherein the water supplied from the feed water inlet is retained in the water retention region by the moisture permeable and water resistant film, an inspiratory gas and an expiratory gas pass through the heat and moisture exchanger element loaded in the aeration region, and the artificial nose carries out a first heating and humidifying process of the inspiratory gas in which heat and moisture included in the expiratory gas passing therethrough are captured and retained by the heat and moisture exchanger element and the heat and the moisture are discharged to an inspiratory gas passing therethrough next, and a second heating and humidifying process in which only water vapor generated by heating of the heater passes through the moisture permeable and water resistant film and is supplied to the inspiratory gas passing through the heat and moisture exchanger element to heat and humidify the inspiratory gas and also the inspiratory gas in the heat and moisture exchanger element is heated by the heater.

Here, the “heat and moisture exchanger element” is a material that captures and retains heat and moisture and further is capable of discharging the heat and the moisture, and as described later, can also be configured with, for example, hygroscopic paper and can also be configured with a resin made foam, a member of resin fibers tangled like cotton wool, or the like.

According to this embodiment, the second heating and humidifying process that supplies heat and moisture to the inspiratory gas by the water vapor permeated from the water retention region, and at the same time, supplies further heat to the inspiratory gas from the heater enables to make up for the heating and humidification of the inspiratory gas insufficient only by the first heating and humidifying process with the heat and moisture exchanger element to realize heating and humidification of the inspiratory gas sufficient for a user. Further, in this embodiment, only the water vapor generated by the heating of the heater passes through the moisture permeable and water resistant film, so that there is no possibility of obstructing the flow channel of the inspiratory gas and the expiratory gas by supplying excessive moisture to the heat and moisture exchanger element and there is no risks of flowing excessive moisture into a trachea or a lung of a user, so that it is possible to realize humidification and heating sufficient for a user in a state of securing safety. Further, the artificial nose is warmed by a heat source of the heater along an outer circumference of the artificial nose, so that the artificial nose itself can be less affected by the external temperature (influence due to the room temperature and an air from an air conditioner or the like) or the like to maintain stable heating and humidification.

This embodiment includes not only a case in which the region filled with the heat and moisture exchanger element coincides with the region provided with the water retention region and the heater but also a case in which, for example, the water retention region and the heater are provided in a region where the heat and moisture exchanger element is not loaded, that is, a case of heating and humidifying the inspiratory gas passing through the aeration region not via the heat and moisture exchanger element.

Another embodiment of an artificial nose of the present invention used for a breathing circuit is the artificial nose, wherein the heater is configured with a wire heater that wraps around outside the outer shell in a region where the water retention region is formed.

According to this embodiment, the heater is disposed in a region where the water retention region is formed, so that the water stored in the water retention region can be heated sufficiently to generate water vapor, and further, the inspiratory gas can be heated and humidified using a sufficient humidifying area corresponding to the water retention region. Similarly, the inspiratory gas can be heated using a sufficient heating area corresponding to the humidifying area.

In addition, a wire heater is wrapped around, thereby allowing a heater to be easily disposed outside the outer shell.

Another embodiment of an artificial nose of the present invention used for a breathing circuit is the artificial nose, wherein the heater is configured with a plate heater disposed outside the outer shell in a region where the water retention region is formed.

According to this embodiment, the heater is disposed in a region where the water retention region is formed, so that the water stored in the water retention region can be heated sufficiently to generate water vapor, and further, the inspiratory gas can be heated and humidified using a sufficient humidifying area corresponding to the water retention region. Similarly, the inspiratory gas can be heated using a sufficient heating area corresponding to the humidifying area.

In addition, a plate heater is disposed outside the outer shell, thereby allowing the water retention region and the aeration region to be heated efficiently.

Another embodiment of an artificial nose of the present invention used for a breathing circuit is the artificial nose, wherein the heating and humidification of the inspiratory gas is possible to be adjusted at the same time by adjusting a power application to the heater.

Suppose if the flow rate of the inspiratory gas flowing in the aeration region increases, an amount of water vapor and an amount of heat to be added to the inspiratory gas are required to increase, and on the contrary, if the flow rate of the inspiratory gas decreases, an amount of water vapor and an amount of heat to be added to the inspiratory gas are required to be reduced. That is, the amount of water vapor and the amount of heat to be added to the inspiratory gas have positive correlation. Accordingly, as this embodiment, the heating and humidification of the inspiratory gas can be adjusted at the same time by adjusting a power application of one heater, and thus the device configuration and the control process can be simplified.

Another embodiment of an artificial nose of the present invention used for a breathing circuit is the artificial nose, wherein the moisture permeable and water resistant film includes a resin made sheet or a resin made film.

According to this embodiment, a resin material is used, thereby a highly reliable moisture permeable and water resistant film can be obtained.

Another embodiment of an artificial nose of the present invention used for a breathing circuit is the artificial nose, wherein the moisture permeable and water resistant film includes a nonwoven fabric having moisture permeability and water resistance.

Here, “the moisture permeable and water resistant film includes a nonwoven fabric having moisture permeability and water resistance” includes a case of using a nonwoven fabric only and also includes a case of using a material having a nonwoven fabric and another member, such as a water absorbing polymer, for example, in combination. According to this embodiment, a film can be obtained that has sufficient moisture permeability and water resistance at relatively low production costs.

Another embodiment of an artificial nose of the present invention used for a breathing circuit is, further, the artificial nose, wherein the moisture permeable and water resistant film includes a porous material or a nonporous material.

Here, a porous material is a material having micropores that is not permeable to a water droplet but permeable to a gas, including water vapor. In contrast, a nonporous material does not have micropores permeable to a gas, a liquid, and a gas, and for example, moisture permeates the material from the surface in contact with a water droplet and diffuses therein and evaporates from the other surface, thereby exhibiting the moisture permeable and water resistant performance.

According to this embodiment, both a porous material and a nonporous material can be used as the moisture permeable and water resistant film, so that it is possible to select an optimal one as the moisture permeable and water resistant film from diverse materials.

Another embodiment of an artificial nose of the present invention used for a breathing circuit is the artificial nose, wherein the heat and moisture exchanger element is configured with a resin made foam, a resin fiber tangled like cotton wool, or hygroscopic paper.

According to this embodiment, various materials can be used as the heat and moisture exchanger element.

In a case that the heat and moisture exchanger element is configured with a resin made foam or a resin fiber, a heat and moisture exchanger element high in reliability and durability can be provided, and in a case that the heat and moisture exchanger element is configured with hygroscopic paper, a heat and moisture exchanger element can be provided at low costs. It is preferred to use an optimal material in accordance with a status of use.

Another embodiment of an artificial nose of the present invention used for a breathing circuit is, further, the artificial nose, wherein a tubular reinforcement member is disposed on the internal surface side of the moisture permeable and water resistant film to make contact with the internal surface.

Here, to be “tubular” is a tubular shape having a hollow inside and includes those having any cross-sectional shape including circular, elliptical, and polygonal shapes. Regarding an aspect ratio (for example, a ratio of a diameter of a cross-section and a longitudinal length), those having any profile is included.

According to this embodiment, even in a case a tube configured with a moisture permeable and water resistant film does not have the strength for maintaining a shape (for example, cylindrical shape) of securing the aeration region, a tubular reinforcement member is disposed so as to make contact with an internal surface of the moisture permeable and water resistant film, so that the tube configured with a moisture permeable and water resistant film can be maintained in the shape and the moisture permeable and water resistant film can be prevented from expanding inward to secure the aeration region in a sufficient size.

The cross-sectional shape of the aeration region secured by the tubular reinforcement member is not limited to a circular shape and can have any cross-sectional shape, including elliptical and polygonal shapes.

Another embodiment of an artificial nose of the present invention used for a breathing circuit is, further, the artificial nose, wherein a helical core is disposed in the water retention region between the outer shell and the moisture permeable and water resistant film and the water supplied from the feed water inlet flows along a helical flow channel formed with the helical core.

According to this embodiment, even in a case that a tube configured with a moisture permeable and water resistant film does not have the strength for maintaining a shape (for example, cylindrical shape) of securing an aeration region, a helical core is disposed in the water retention region, so that the tube configured with a moisture permeable and water resistant film can be maintained in the shape and the moisture permeable and water resistant film can be prevented from expanding inward to secure the aeration region in a sufficient size. Since water flows along a helical flow channel formed with the helical core, the helical core does not impede the flow of the water in the water retention region.

The cross-sectional shape of the aeration region secured by the helical core is not limited to a circular shape and can have any cross-sectional shape, including elliptical and polygonal shapes.

One embodiment of a breathing circuit of the present invention is a breathing circuit, including: the above artificial nose; an inspiratory tube and an expiratory tube in communication with one end of the aeration region of the artificial nose; an inspiratory gas supply source supplying the inspiratory gas to the inspiratory tube; and water supply means supplying the water to the water retention region with an approximately constant static pressure via the feed water inlet, wherein the water retention region is supplemented with water by the water supply means in an amount of water corresponding to an amount of water vapor passed through the moisture permeable and water resistant film and flown out.

According to this embodiment, in addition to actions and effects included in the above artificial nose, by applying an approximately constant static pressure, the water retention region can be supplemented with water in an amount of water corresponding to the amount of water vapor that has gone out through the moisture permeable and water resistant film, so that a breathing circuit can be provided that is capable of heating and humidifying an inspiratory gas stably for a long period of time without an excessive control or the like.

Another embodiment of a breathing circuit of the present invention is the breathing circuit, wherein the water supply means supplies the water by dropping from a container that contains the water and includes: drop rate measurement means measuring a rate of the dropping; and control means carrying out a control process of issuing an alert, based on drop rate measurement data sent from the drop rate measurement means, when the drop rate exceeds a predetermined value or when the drop rate falls below a predetermined value.

According to this embodiment, a control process of issuing an alert is carried out when the drop rate from the container containing water exceeds a predetermined value, so that even if the moisture permeable and water resistant film is broken to cause an event of water leakage, it is possible to secure safety of the user by issuing an alert promptly. A control process of issuing an alert is also carried out when the drop rate from the container containing water falls below a predetermined value, so that even in a case that the water supply tank becomes empty or that water becomes not supplied to the artificial nose for some reason (for example, an obstruction of the tube), it is possible to secure safety of the user by issuing an alert promptly.

### Effect of the Invention

As described above, in an artificial nose of the present invention and a breathing circuit provided with the artificial nose, the second heating and humidifying process that supplies heat and moisture to the inspiratory gas by the water vapor permeated from the water retention region, and at the same time, supplies further heat to the inspiratory gas from the heater enables to make up for the heating and humidification of the inspiratory gas insufficient only by the first heating and humidifying process with the heat and moisture exchanger element to realize heating and humidification of the inspiratory gas sufficient for a user. Further, only the water vapor generated by the heating of the heater passes through the moisture permeable and water resistant film, so that there is no possibility of obstructing the flow channel of the inspiratory gas and the expiratory gas by supplying excessive moisture to the heat and moisture exchanger element and there is no risks of flowing excessive moisture into a trachea or a lung of a user, so that it is possible to realize humidification and heating sufficient for a user in a state of securing safety. Further, the artificial nose is warmed by a heat source of the heater along an outer circumference of the artificial nose, so that the artificial nose itself can be less affected by the external temperature (influence due to the room temperature and an air from an air conditioner or the like) or the like to maintain stable heating and humidification.

### BRIEF DESCRIPTION OF THE DRAWINGS

FIG. 1 is a full view of one embodiment of an artificial nose of the present invention used for a breathing circuit.

FIGS. 2(a) and 2(b) are schematic views illustrating internal structures of a first embodiment of the artificial nose shown in FIG. 1.

FIG. 3 is a schematic view illustrating an internal structure of a second embodiment of an artificial nose of the present invention.

FIGS. 4(a) and 4(b) are schematic views illustrating internal structures of a third embodiment of an artificial nose of the present invention.

FIG. 5 is a schematic view illustrating a configuration of a breathing circuit provided with an artificial nose of the present invention.

FIG. 6 is a diagram schematically illustrating structures of a porous material and a nonporous material.

FIG. 7 is a schematic view illustrating a structure of an embodiment of an artificial nose using a nonporous material as a moisture permeable and water resistant film.

FIG. 8 is a schematic view illustrating a structure of an embodiment of an artificial nose having a tubular reinforcement member disposed therein so as to make contact with an internal surface of a moisture permeable and water resistant film.

FIG. 9 is a schematic view illustrating a structure of an embodiment of an artificial nose having a helical core disposed therein in a water retention region between an outer shell and a moisture permeable and water resistant film.

FIG. 10 is a schematic view illustrating an internal structure of a conventional artificial nose.

### DETAILED DESCRIPTION OF THE INVENTION

Embodiments of an artificial nose of the present invention used for a breathing circuit are described below with reference to the drawings.

### Description of First Embodiment of Artificial Nose According to the Invention

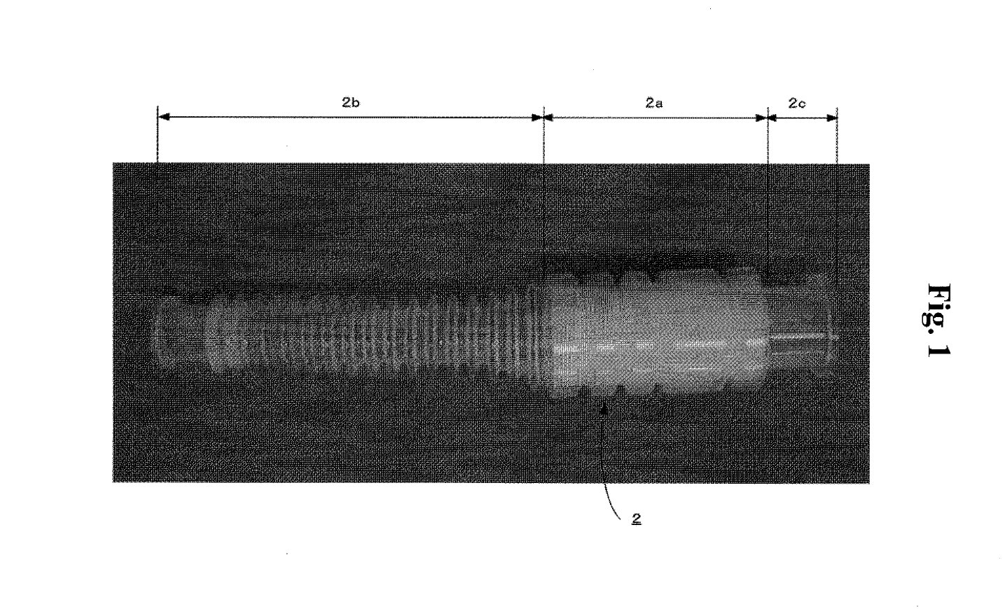

FIG. 1 is a full view (photograph) of a first embodiment of an artificial nose according to the present invention. An artificial nose 2 of the present embodiment is configured with an artificial nose main body 2a, and a user side end 2b and an inspiratory gas supply source side end 2c that are integrally formed with both ends thereof.

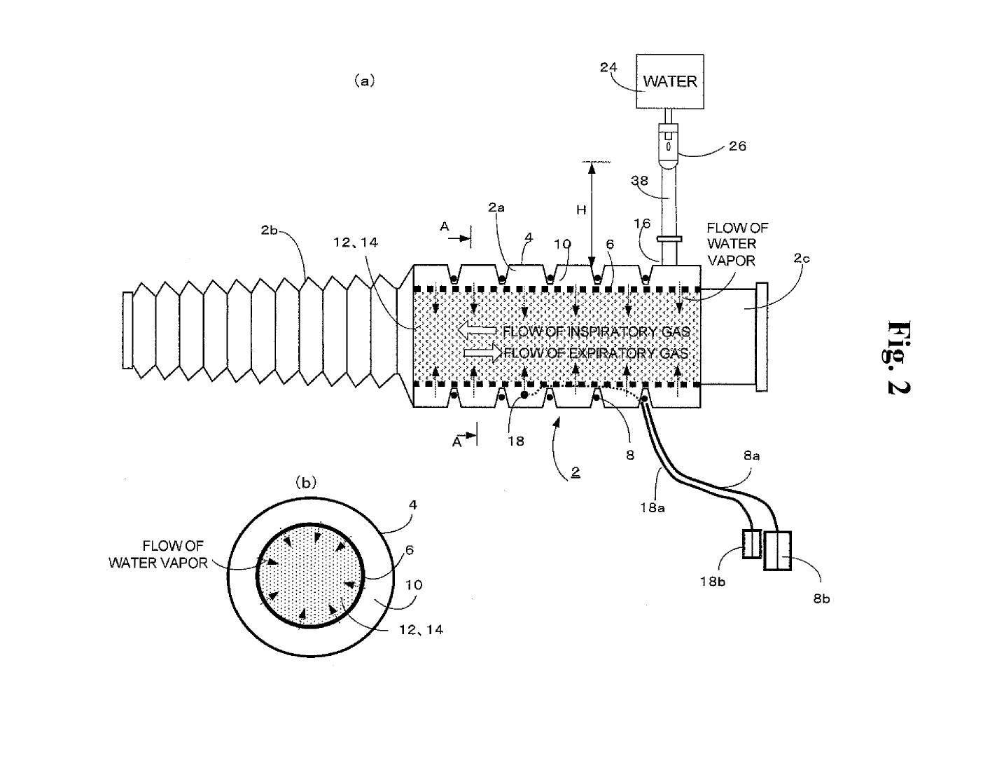

FIGS. 2(a) and 2(b) are schematic views illustrating internal structures of a first embodiment of the artificial nose shown in FIG. 1. FIG. 2(a) is a schematic view of the artificial nose 2 taken from a side, where the area of the artificial nose main body 2a is illustrated a state of eliminating an outer shell 4 to expose inside thereof. FIG. 2(b) is a cross-sectional view taken from arrows A-A in FIG. 2(a).

An artificial nose main body 2a is provided with a cylindrically shaped outer shell 4 having air tightness and water tightness, a moisture permeable and water resistant film 6 having moisture permeability and water resistance disposed on the entire circumference of the internal surface of the outer shell 4, a heat and moisture exchanger element 14 mounted inside the moisture permeable and water resistant film 6, and a wire heater 8 wrapped around outside the outer shell 4. Thus, a water retention region 10 is formed between the internal surface of the outer shell 4 and an outer surface of the moisture permeable and water resistant film 6, and an aeration region 12 is formed on the internal surface side of the moisture permeable and water resistant film 6. That is, the water retention region 10 and the aeration region 12 are partitioned by the moisture permeable and water resistant film 6. Then, the heat and moisture exchanger element 14 is loaded in this aeration region 12. Here, the heat and moisture exchanger element 14 is a material that captures and retains heat and moisture and further is capable of discharging the captured and retained heat and the moisture. Although the heat and moisture exchanger element 14 of the present embodiment is configured with a resin made foam, it may also be configured with a resin fiber tangled like cotton wool (for example, like nylon wool). The heat and moisture exchanger element 14 using such a resin material is excellent in reliability and durability. In addition, it may also be configured with, for example, hygroscopic paper, and in this case, the heat and moisture exchanger element 14 can be provided at low costs. As described above, it is preferred to use an optimal material in accordance with a status of use.

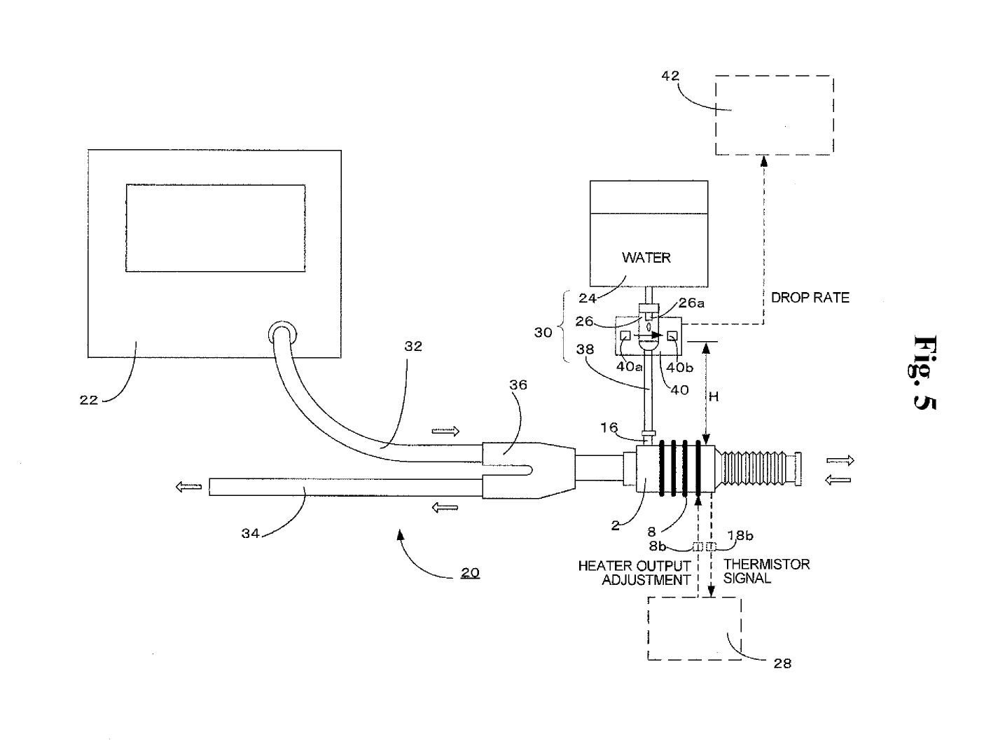

The outer shell 4 is formed integrally with a feed water inlet 16 (not shown in FIG. 1), and a water supply tube 38 is connected to this feed water inlet 16. As shown in FIG. 2(a), water supplied from a water container 24 is led into the water retention region 10 from a feed water inlet 16 through a dropping chamber 26 and the water supply tube 38 (the dropping chamber 26 is described later in detail using FIG. 5). In this case, water is supplied to the water retention region 10 with a static pressure of a head of water H (difference between the water surface of the dropping chamber 26 and the height of the water retention region 10). The outer shell 4 has air tightness and water tightness and the moisture permeable and water resistant film 6 has moisture permeability and water resistance which is permeable to a gas, like water vapor, but not permeable to water, which is a liquid, so that the water supplied from the feed water inlet 16 is retained in the water retention region 10 formed between the outer shell 4 and the moisture permeable and water resistant film 6.

The wire heater 8 of the present embodiment is a resistive heating wire heater (so-called ribbon heater) and wraps around an outer surface of the outer shell 4 in the entire region where the water retention region 10 is formed.

The artificial nose 2 with a configuration as mentioned above has, as shown in FIG. 5, one end in communication with an inspiratory tube 32 and with an expiratory tube 34 via a Y shaped connector 36 and has the other end connected to an intratracheal tube of the user. This intratracheal tube is inserted to a patient from the nose (in a case of nasal intubation), the mouth (in a case of oral intubation), or the trachea (in a case of tracheal intubation). In addition, the inspiratory tube 32 is connected to an inspiratory supply source 22. Therefore, an inspiratory gas at a predetermined flow rate is supplied to the inspiratory tube 32 by the inspiratory supply source 22, and the inspiratory gas passes through the inspiratory tube 32 and the Y shaped connector 36 and flows in the aeration region 12 of the artificial nose 2 to be supplied to the user. The expiratory gas exhaled from the user flows in the aeration region 12 of the artificial nose 2 and passes through the Y shaped connector 36 and the expiratory tube 34 to be discharged to the atmosphere.

In FIG. 2(a), as shown with a hollow arrow, the inspiratory gas flows in the aeration region 12 of the artificial nose main body 2a from the right side to the left side of the drawing, and the expiratory gas flows in the aeration region 12 of the artificial nose main body 2a from the left side to the right side of the drawing. Although the outer shell 4 is normally in a cylindrical shape having a circular cross-sectional shape, it is not limited thereto and a case is also possible that has, for example, an elliptical or polygonal cross-sectional shape.

In the present embodiment, the heat and moisture exchanger element 14 is loaded in the entire region of the aeration region 12 of the artificial nose main body 2a. The expiratory gas has a temperature of more or less 37° C. and a relative humidity of 100%, and the heat and the moisture included in the expiratory gas exhaled from the user can be captured and retained by the heat and moisture exchanger element 14. Then, the captured and retained heat and moisture are discharged to an inspiratory gas that flows in the heat and moisture exchanger element 14 next to carry out a first heating and humidifying process of heating and humidifying the inspiratory gas.

However, the heating and humidification of the inspiratory gas cannot be carried out sufficiently only in the first heating and humidifying process. With that, in the present embodiment, a second heating and humidifying process is carried out in which heat and moisture is supplied to the inspiratory gas by the water vapor permeated from the water retention region 10, and at the same time, further heat is supplied to the inspiratory gas from the heater 8.

That is, a predetermined power is supplied to the wire heater 8 in a state where water is retained in the water retention region 10, thereby heating the water retained in the water retention region 10 to generate water vapor. The generated water vapor permeates the moisture permeable and water resistant film 6 as shown with arrows in broken lines in FIGS. 2(a) and 2(b) and flows into the heat and moisture exchanger element 14 loaded in the aeration region 12 to be supplied to the inspiratory gas passing through the heat and moisture exchanger element 14. Thus, the inspiratory gas can be heated and humidified.

At the same time to this, the wire heater 8 can give not only the water in the water retention region 10 but also a predetermined amount of heat to the inspiratory gas passing through the heat and moisture exchanger element 14 in the aeration region 12, so that the inspiratory gas can also be heated. As described above, in addition to the first heating and humidifying process by the heat and moisture exchanger element 14, the second heating and humidifying process that supplies heat and moisture from the water retention region 10 to the inspiratory gas, and at the same time, applies further heat to the inspiratory gas with the wire heater 8 enables to realize humidification and heating of the inspiratory gas sufficient for a user.

In the present embodiment, by the wire heater 8, the inspiratory gas can be heated and humidified at the same time. Suppose if the flow rate of the inspiratory gas flowing in the aeration region 12 increases, the amount of water vapor and the amount of heat to be added to the inspiratory gas is required to be increased, and if the flow rate of the inspiratory gas decreases, the amount of water vapor and the amount of heat to be added to the inspiratory gas is required to be reduced. That is, the amount of water vapor and the amount of heat to be added to the inspiratory gas have positive correlation. Accordingly, as the present embodiment, the heating and humidification of the inspiratory gas can be adjusted at the same time by adjusting the power application of the one heater 8, and thus the device configuration and the control process can be simplified.

In the present embodiment, the wire heater 8 is disposed outside the outer shell 4 in the entire region where the water retention region 10 is formed. This enables the water stored in the water retention region 10 to be heated sufficiently to generate water vapor, and further, to heat and humidify the inspiratory gas passing through the heat and moisture exchanger element 14 in the aeration region 12 using the sufficient humidifying area corresponding to the water retention region 10. Similarly, using the sufficient heating area corresponding to the humidifying area, the inspiratory gas passing through the heat and moisture exchanger element 14 in the aeration region 12 can be heated.

A detailed description is give below to components configuring the artificial nose 2 of the present embodiment.

### <Description of Outer Shell 4>

The outer shell 4 is configured with a resin material having air tightness and water tightness and also flexibility, and in the present embodiment, it is configured with vinyl chloride. It should be noted that it is not limited thereto and any other resin material, including polypropylene, polyethylene, polyethylene and ethylene vinyl acetate, and polyvinyl chloride, can be used.

In addition, with the outer shell 4, the artificial nose main body 2a, and the user side end 2b and the inspiratory gas supply source side end 2c are integrally formed with both ends thereof.

The outer shell 4 of the present embodiment is formed with a recess and a protrusion, and the wire heater 8 wraps around the recess. The heater 8 of the present embodiment is configured with one wire heater, and although not shown, the wire heater 8 wrapping around each recess is joined to each other by the wire heater 8 extending laterally in the drawing. The recess can also be formed helically to wrap the wire heater 8 around the outer surface of the outer shell 4 along this helical recess.

In the present embodiment, the heater 8 is mounted in the recess of the outer shell 4, so that there is no possibility of burning even when the artificial nose 2 is touched with a bare band or touches the skin of the patient. This recess on the outer circumference of the outer shell 4 also serves as a reinforcing member to increase the strength not to easily collapse the column of the artificial nose 2. The user side end 2b of the artificial nose 2 is designed to move the tube flexibly to easily attach a tip of the user side end 2b to the patient. The present invention also includes an artificial nose 2 not having the user side end 2b, and in this case, it is preferred to use the artificial nose 2 by loading a flexible tube, which is a separate member.

Such a configuration as above enables the wire heater 8 to be disposed evenly on the entire circumference of the outer shell 4 of the water retention region 10. This enables to realize even heating of the water and the inspiratory gas in the entire area of the water retention region 10. It should be noted that the shape of the outer surface of the outer shell 4 is not limited thereto and it can also have a flat outer surface with no recess and protrusion.

### <Description of Moisture Permeable and Water Resistant Film 6>

The moisture permeable and water resistant film 6 of the present embodiment is configured with a moisture permeable and water resistant sheet or a moisture permeable and water resistant film, and can be formed by rolling this sheet/film in a tubular shape to a diameter slightly smaller than the inner diameter of the outer shell 4 and seal bonding the both ends in the total longitudinal length. This moisture permeable and water resistant film 6 in a tubular shape is inserted into the outer shell 4 of the artificial nose main body 2a and this outer shell 4 and the moisture permeable and water resistant film 6 are seal bonded at the both longitudinal ends of the outer shell 4, thereby enabling to form the structure shown in FIG. 2(a). These seal bondings can be realized using an adhesive.

The static pressure (for example, head of water H=100 cm H₂O) applied to the water retention region 10 is not high, so that the moisture permeable and water resistant film 6 is considered to obtain sufficient rigidity by bonding at the both longitudinal ends of the outer shell 4 of the artificial nose main body 2a while it is also possible to spot bond the outer shell 4 and the moisture permeable and water resistant film 6 with a predetermined pitch as needed.

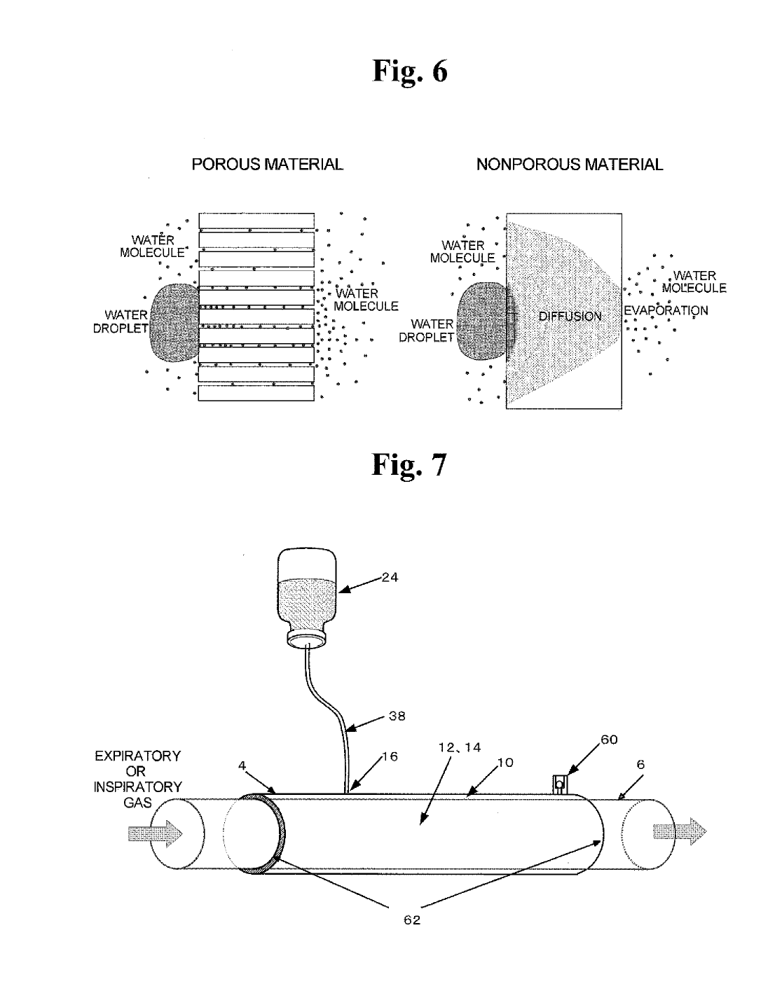

The moisture permeable and water resistant sheet/film used for the moisture permeable and water resistant film 6 is required to have a moisture permeable performance that is sufficiently permeable to water vapor and a water pressure resistant performance that can sufficiently withstand the applied water pressure. As a moisture permeable and water resistant sheet/film requiring such performances, porous materials and nonporous materials as shown in FIG. 6 can be used.

As shown in a left drawing of FIG. 6, a porous material is a material having micropores that are not permeable to a water droplet but permeable to a gas, and the micropores are permeable to water vapor, which is a gas including water molecules. An amount of permeating water vapor is determined by a humidity difference and a temperature difference between the spaces on both sides interrupted by the porous material. That is, in the left drawing of FIG. 6, in a case that the humidity is low and the temperature is high in the right side region of the porous material, the amount of permeating water vapor increases.

Such a structure enables to have the moisture permeable performance that is sufficiently permeable to water vapor and the water pressure resistant performance that can sufficiently withstand the applied water pressure. Specific examples of a porous material may be the materials shown in Table 1 described later.

In contrast, as shown in a right drawing of FIG. 6, a nonporous material does not have the micropores that are permeable to liquids gases, and moisture permeates the material from the surface in contact with a water droplet and diffuses therein and evaporates from the other surface, thereby exhibiting a moisture permeable and water resistant performance. The amount of permeating water vapor is determined by a temperature difference between the spaces on the both sides interrupted by the porous material. That is, in the right drawing of FIG. 6, in a case that the temperature in the right side region of the porous material is high, the amount of permeating water vapor increases.

Such a structure enables a nonporous material to have the moisture permeable performance that is sufficiently permeable to water vapor and the water pressure resistant performance that can sufficiently withstand the applied water pressure. Specific examples of a nonporous material may be a moisture permeable and water resistant sheet/film supplied by ARKEMA and a moisture permeable and water resistant sheet/film called SYMPATEX, a trade name, supplied by Akzo Nobel.

FIG. 7 illustrates an embodiment of the artificial nose 2 in a case of using a nonporous material as the moisture permeable and water resistant film 6 This artificial nose 2 is provided with the tubular outer shell 4 having air tightness and water tightness and the moisture permeable and water resistant film 6 including a nonporous material disposed on the entire circumference of the internal surface of the outer shell 4, and at both ends, the outer shell 4 and the moisture permeable and water resistant film 6 are seal bonded by a sealing member 62. Thus, the water retention region 10 is formed between the internal surface of the outer shell 4 and the outer surface of the moisture permeable and water resistant film 6, and the aeration region 12 (the heat and moisture exchanger element 14 is filled inside) is formed on the internal surface side of the moisture permeable and water resistant film 6.

To be “tubular” is a tubular shape having a hollow inside and includes those having any cross-sectional shape (circular shape in FIG. 7) including circular, elliptical, and polygonal shapes. Regarding an aspect ratio (for example, a ratio of a diameter of a cross-section and a longitudinal length), those having any profile is included, and not only the profiles as shown in FIG. 7 but also the profiles as shown in FIGS. 1 through 5 are also included.

The water stored in the water container 24 is led into the water retention region 10 from the feed water inlet 16 through the water supply tube 38. At this time, to make the water flow into the water retention region 10, it is required to exhaust the air present in the water retention region 10 to outside the water retention region 10 in advance. In this case, if the moisture permeable and water resistant film 6 were a porous material, the air could be exhausted through the micropores of the porous material, while if the moisture permeable and water resistant film 6 is a nonporous material, exhaustion cannot be carried out through the moisture permeable and water resistant film 6.

With that, the embodiment shown in FIG. 7 is provided with an exhaust outlet 60 to exhaust the air present in the water retention region 10 in advance via the exhaust outlet 60. This exhaust outlet 60 is provided with a check valve, which allows exhausting the air in the water retention region 10 but does not allow the external air to flow into the water retention region 10. Although FIG. 7 shows a ball check valve, it is not limited thereto and can use any other types of check valve.

In the present embodiment, by capping the exhaust outlet 60 after exhausting all air in the water retention region 10, the water in the water retention region 10 is kept from flowing out to outside. It should be noted that it is not limited thereto and the exhaust outlet 60 to flow the air but not to flow water can also be formed by, for example, putting a porous material on a top opening of the exhaust outlet 60.

It is also possible to put a highly hygroscopic material, such as a gel water absorbing and filter paper, for example, in the water retention region 10 formed between the outer shell 4 and the moisture permeable and water resistant film 6.

As described above, in the present embodiment, not only a porous material but also a nonporous material can be used as the moisture permeable and water resistant film 6 by being provided with the exhaust outlet 60, so that it is possible to select an optimal one as the moisture permeable and water resistant film 6 from diverse materials.

Next, the moisture permeable performance (degree of moisture permeability) and the water pressure resistant performance (water pressure resistance) required as the moisture permeable and water resistant film 6 are reviewed as below.

Ideal heating and humidifying conditions required for an artificial nose or anesthesia are generally to supply an inspiratory gas having a relative humidity of 100% (44 mg /L maximum) at a temperature of 37° C. to a user. Meanwhile, the maximum amount of water that can be transferred from the expiratory gas to the inspiratory gas by the heat and moisture exchanger element 14 is 30 mg/L. Accordingly, it is required for the water vapor having permeated the moisture permeable and water resistant film 6 to supply deducted moisture of 14 mg/L (=44−30) to the inspiratory gas.

Therefore, where an amount of breathing of an adult male is 6 L/min, the maximum amount of water vapor to be supplied to the inspiratory gas by permeating the moisture permeable and water resistant film 6 for 24 hours becomes:

6 (L/min)×14 (mg/L)×60×24×1/1000=approximately 121 g/24 hrs.

A humidifying area to make water vapor permeate (area of the moisture permeable and water resistant film 6) is considered to be, assuming that, for example, the water retention region 10 has an inner diameter of 3 cm and has a length of 20 cm, approximately 0.019 m²(=3/100×20/100×3.14).

Accordingly, 121 g/24 hrs of water vapor is required to permeate in the entire area of the moisture permeable and water resistant film 6 having a humidifying area of 0.019 m², so that a degree of moisture permeability of approximately 6,368 g/m2·24 hr (=121/0.019) is required for a moisture permeable and water resistant sheet/film used for the moisture permeable and water resistant film 6.

Then, the water pressure resistant performance (water pressure resistance) of the moisture permeable and water resistant film 6 is reviewed where the dimensions of H shown in FIG. 2(a) is considered to be approximately from 40 cm to 200 cm by considering specific arrangement of the artificial nose 2 and water supply means 30. Accordingly, 200 cm H₂O or more of water pressure resistance is considered to be required.

The moisture permeable performance required for actual use is, taking a safety factor of some extent into consideration, a degree of moisture permeability (JIS K 7129 (A method)) of preferably 7,000 g/m2·24 hr or more, more preferably 10,000 g/m2·24 hr or more, and even more preferably 12,000 g/m2·24 hr.

The water pressure resistance is, taking a safety factor of some extent into consideration, preferably 400 cm H₂O or more, more preferably 800 cm H₂O or more, and even more preferably 1000 cm H₂O or more.

Some examples of a specific material (porous material) having such a moisture permeable performance and a water pressure resistant performance are shown in the table below. In the table below, materials including resinous sheets/films and a nonwoven fabric are shown.

TABLE 1

| | | | Degree Of | | |
|---|---|---|---|---|---|
| | | | Moisture | Water | |
| | | | Permeability | Pressure | |
| | | Corporate | A Method | Resistance | |
| NO | Trade Name | Name | g/m2 · 24 hr | cmH₂O | Material |
| 1 | FGX Film | Hiramatsu | 14,000 | 3,000 | Polyurethane |
| | | Sangyo | | | Porous Film |
| | | Company | | | |
| 2 | GEOVISOR-αD | Toyocloth | 10,000 | 499 | Urethane |
| | | Co., Ltd | | | |
| 3 | AGX-3381 | Toyocloth | 10,240 | 1,200 | Polyurethane |
| | | Co., Ltd. | | | |
| 4 | Gore-Tex XCR | Japan | 13,500 | 4,000 | Teflon |
| | | Gore-Tex Inc. | | | |
| 5 | Microporous Film | Sumitomo 3M | 12,000 | 1,000 | Polypropylene |
| | | Limited | | | based |
| | | | | | Microporous |
| | | | | | Film |
| 6 | Mitsubishi | EXEPOL | 7,200 | 1,600 | Polyethylene |
| | Plastics, Inc. | | | | |

In a case of using a resinous material having the moisture permeable performance and the water pressure resistant performance (for example, the materials of from #1 to #5 in Table 1), it is possible to obtain a highly reliable moisture permeable and water resistant film 6 In a case of using a nonwoven fabric, it is possible to obtain a moisture permeable and water resistant film 6 at relatively low production costs. Since there is a possibility of large water leakage, once water permeates, from that spot in a case of a nonwoven fabric singly, it is preferred to use a material, for example, having a nonwoven fabric and a water absorbing polymer or the like in combination (for example, the material of #6 in Table 1).

It should be noted that the material including a moisture permeable and water resistant sheet/film and a nonwoven fabric used for the moisture permeable and water resistant film 6 is not limited to the materials including the resinous sheets/films and the nonwoven fabric mentioned above, and it is possible to use a material including any resinous sheet/film and nonwoven fabric having a predetermined moisture resistant performance and a predetermined water pressure resistant performance.

### <Description of Heater 8>

In the present embodiment a so-called ribbon heater (a nichrome wire coated by a fabric woven with heat resistant glass fibers) is used as the heater 8, so that it is excellent in flexibility and can easily wrap around along the recess on the outer surface of the outer shell 4.

A method of embedding a heater wire 8 of a thin nichrome wire in the outer shell 4 is also considered. In this case, it is formed by pasting two sheets of a member configuring the outer shell 4 together to put the heater wire 8 between the pasted sheets. This also enables insulation, so that it is possible to provide an artificial nose 2 having the outer shape, the weight, and the usability almost not different from those of an artificial nose having no heater wire 8.

In the embodiment shown in FIGS. 2(a) and 2(b), the wire heater 8 is connected to a heater cable 8a, and at the other end of the heater cable 8a, a heater power connector 8b is connected for termination. In the water retention region 10, a thermistor 18 is arranged, and this thermistor 18 is connected to a thermistor cable 18a, and at the other end of the thermistor cable 18a, a thermistor connector 18b is connected for termination. Then, as shown in FIG. 5, the heater power connector 8b and the thermistor connector 18b are connected to heater output adjustment means 42, respectively.

Here, a thermistor is one type of a temperature sensor element made with an oxide semiconductor material having a varying resistance value depending on the temperature, and in the present embodiment, based on the temperature measurement data by the thermistor 18, the power application to the heater 8 is adjusted by the heater output adjustment means 42 to keep the temperature in the water retention region 10 always at 40° C. This enables to realize optimal heating and humidification.

As shown in FIG. 2(a), the wire heater 8 is disposed in a region where the water retention region 10 is formed, so that the water stored in the water retention region 10 can be heated sufficiently to generate water vapor, and further, the inspiratory gas passing through the heat and moisture exchanger element 14 in the aeration region 12 can be heated and humidified using the sufficient humidifying area corresponding to the water retention region 10. Similarly, using the sufficient heating area corresponding to the humidifying area, the inspiratory gas passing through the heat and moisture exchanger element 14 in the aeration region 12 can be heated. This enables to make up for the heat and the moisture insufficient by the heating and humidification with the heat and moisture exchanger element 14 and to supply an inspiratory gas at a temperature of 37° C. and a relative humidity of 100% to the user.

Then, a specific heating capacity of the heater 8 is reviewed. As the above description, a case of generating water vapor at 121 g/24 hr is considered, assuming that the heat of vaporization of water at 20° C. (water temperature in the water retention region 10) is 586 cal/g and the thermal efficiency of the heater for the power application is 20%, to have the power application required for the heater being 121 (g/24 hrs)×586 (cal/g)×1/24×1/860 (cal/Wh)/0.2=17 W·hr.

Accordingly, taking a safety factor of some extent into consideration, it is considered that sufficient water vapor can be generated by applying power at approximately from 20 to 30 W·hr to the heater 8. In contrast, in a case of heating the inspiratory gas, the specific heat of the inspiratory gas is very low compared to the heat of vaporization of water, so that it is considered that the heating of an inspiratory gas can be covered sufficiently by applying power at approximately from 20 to 30 W·hr to the heater 8. The power applications are merely some examples, and the optimal heater capacity may be determined in accordance with the flow rate of the inspiratory gas and the range of the water retention region that are actually used. Where the flow rate of the inspiratory gas and the range of the water retention region are considered, it is considered to be preferred to provide the heater 8 with a capacity of approximately from 15 to 100 W.

### <Description of Balance of Heating and Humidification>

As the above description, since the amount of water vapor and the amount of heat to be added to the inspiratory gas have positive correlation, the heating and humidification of the inspiratory gas can be adjusted at the same time by adjusting the power application of one wire heater 8 as the present embodiment. However, since the amount of water vapor and the amount of heat to be added to the inspiratory gas cannot be adjusted individually, it is required to adjust the volume of the water retention region 10, the capacity of the wire heater 8, the humidifying area, the heating area, or the like in advance so as to balance the amount of water vapor and the amount of heat. That is, within the range of adjusting power applied to the wire heater 8, it is required to generate heating and humidification at a rate not causing a trouble for actual use.

For example, even with the same humidifying area and the same heating area, when the interval between the outer shell 4 and the moisture permeable and water resistant film 6 are different, the volume of the water retention region 10 changes, so that the amount of generated water vapor becomes different even if the same amount of power (electric power) is applied to the wire heater 8. In a case of intending to increase the ratio of heating to humidification, it is also possible to dispose the heater 8 outside the outer shell 4 in a region where there is no water retention region 10. On the contrary, in a case of intending to increase the ratio of humidification to heating, it is also considered to use a highly thermally insulative material as the moisture permeable and water resistant film 6.

Adjusting various elements as above enables the heating and humidification of the inspiratory gas to be adjusted at the same time with no problem for actual use by adjusting the power application of one wire heater 8.

### <Description of Feed Water Inlet 16>

In the present embodiment, the feed water inlet 16 is integrally formed together with the outer shell 4. It should be noted that it is not limited thereto and it can be formed by making a hole, in the outer shell 4, having a diameter approximately identical to an outer diameter of the tube, by inserting the water supply tube into this hole, and by seal bonding the outer circumference of the tube and the outer shell 4 using an adhesive.

As described above, according to the above embodiment, the second heating and humidifying process that supplies heat and moisture to the inspiratory gas by the water vapor permeated from the water retention region 10, and at the same time, supplies further heat to the inspiratory gas from the heater enables to make up for the heating and humidification of the inspiratory gas insufficient only by the first heating and humidifying process with the heat and moisture exchanger element 14 to realize heating and humidification of the inspiratory gas sufficient for a user. Further, in this embodiment, only the water vapor generated by the heating of the wire heater 8 passes through the moisture permeable and water resistant film 6, so that there is no possibility of obstructing the flow channel of the inspiratory gas and the expiratory gas by supplying excessive moisture to the heat and moisture exchanger element 14 and there is no risks of flowing excessive moisture into a trachea or a lung of a user, so that it is possible to realize humidification and heating sufficient for a user in a state of securing safety. Further, the artificial nose is warmed by a heat source of the heater along an outer circumference of the artificial nose, so that the artificial nose itself can be less affected by the external temperature (influence due to the room temperature and an air from an air conditioner or the like) or the like to maintain stable heating and humidification.

Although in this embodiment the region loaded with the heat and moisture exchanger element 14 coincides with the region provided with the water retention region 10 and the heater 8, it is not limited thereto, and for example, the water retention region 10 and the wire heater 8 can also be provided in a region where the heat and moisture exchanger element 14 is not loaded. That is, the inspiratory gas passing through the aeration region 12 can also be heated and humidified not via the heat and moisture exchanger element 14. Further, it is also considered only to heat, where there is no water retention region 10 and there is a region in which only the wire heater 8 is mounted outside the outer shell 4, the inspiratory gas passing through the aeration region 12 in this region. The moisture captured and retained by the heat and moisture exchanger element 14 is made into water vapor to be supplied to the inspiratory gas, so that it is preferred to mount the heater 8 for heating outside the outer shell 4 in the region where the heat and moisture exchanger element 14 is loaded.

### Description of Second Embodiment of Artificial Nose According to the Invention

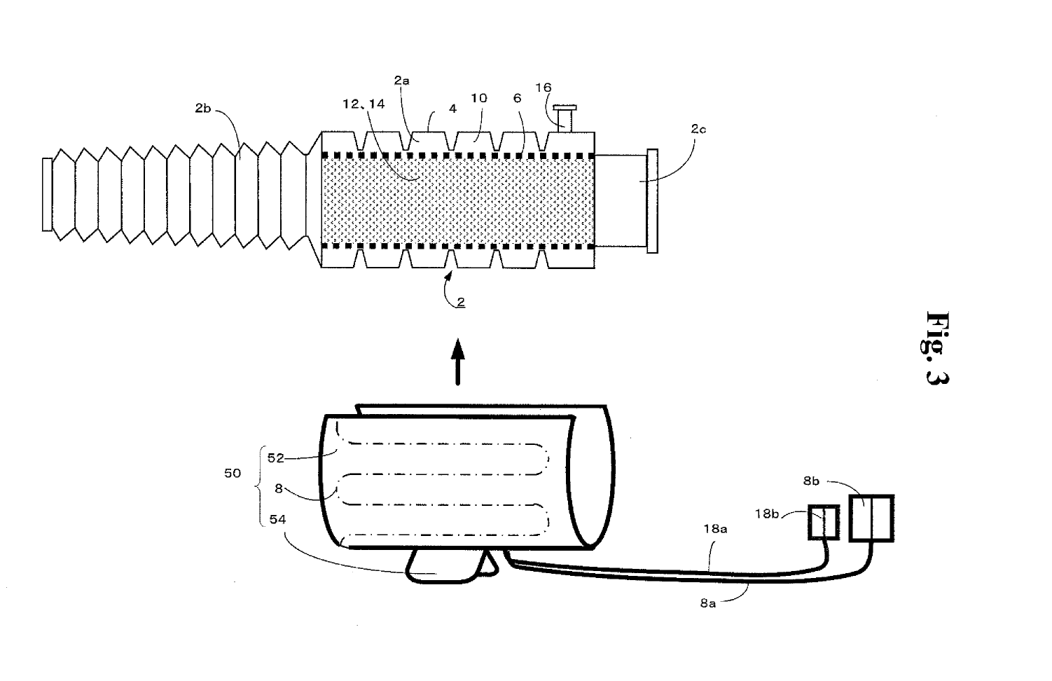

Then, using FIG. 3, a description is given to a second embodiment of an artificial nose according to the present invention. While the heating is carried out by the wire heater 8 in the first embodiment shown in FIGS. 2(a) and 2(b), the present embodiment is different in a point that the heating is carried out by a plate heater 50. Other points in the present embodiment are approximately identical to those in the embodiment shown in FIGS. 2(a) and 2(b), so that only the differences relates to the heater are described below.

The plate heater 50 of the present embodiment is mainly configured with a heater main body 52 including a resin material, the wire heater 8 running throughout a surface portion of the heater main body 52, and a clip 54 attached to the heater main body 52. The plate heater 50 is bent in a cylindrical shape and is biased in a direction of shrinking the inner diameter of the cylinder. Accordingly, by sandwiching the clip 54 with fingers to open the heater main body 52 wound in a cylindrical shape, it is loaded outside the artificial nose main body 2a. Due to the spring force of the clip 54, the internal surface of the heater main body 52 comes into strong contact with the outer surface of the artificial nose main body 2a, so that the heat generated from the wire heater 8 can be transferred efficiently to the artificial nose main body 2a.

In the present embodiment, the plate heater 50 is disposed in the entire region where the water retention region 10 is formed, so that the water stored in the water retention region 10 can be heated sufficiently to generate water vapor, and further, the inspiratory gas passing through the heat and moisture exchanger element 14 in the aeration region 12 can be humidified using the sufficient humidifying area corresponding to the water retention region 10. Similarly, using the sufficient heating area corresponding to the humidifying area, the inspiratory gas passing through the heat and moisture exchanger element 14 in the aeration region 12 can be heated. This enables to make up for the heat and the moisture insufficient by the heating and humidification with the heat and moisture exchanger element 14 and to supply a sufficiently heated and humidified (for example, at a temperature of 37° C. and a relative humidity of 100%) inspiratory gas to the user.

In particular, in the present embodiment, the plate heater 50 is disposed outside the artificial nose main body 2a, thereby enabling the water retention region 10 and the aeration region 12 to be heated efficiently.

### Description of Third Embodiment of Artificial Nose According to the Invention

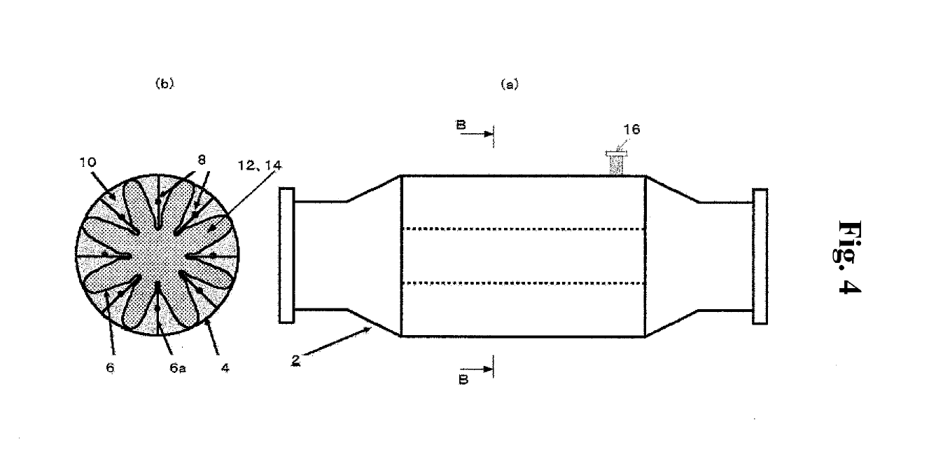

Then, using FIGS. 4(a) and 4(b), a description is given to a third embodiment of an artificial nose according to the present invention. FIG. 4(a) is a full view of the artificial nose 2 taken from the side, and FIG. 4(b) is a cross-sectional view taken from the arrows B-B in FIG. 4(a).

The configuration members of the present embodiment are similar to those of the first embodiment shown in FIGS. 2(a) and 2(b), and is provided with the outer shell 4, the moisture permeable and water resistant film 6 disposed on the entire circumference the an internal surface of the outer shell 4, the wire heater 8, and the heat and moisture exchanger element 14. Then, the water retention region 10 is formed between the outer shell 4 and the moisture permeable and water resistant film 6, and the aeration region 12 is formed on the internal surface side of the moisture permeable and water resistant film 6. In addition, the heat and moisture exchanger element 14 is loaded in the aeration region 12, and the outer shell 4 is provided with the feed water inlet 16 to supply water to the water retention region 10.

What the present embodiment is different from the first embodiment is a point that the moisture permeable and water resistant film 6 is formed in a wavy shape like the folds of a nasal cavity of a person in the cross-sectional shape shown in FIG. 4(b). In order to form the wavy shape, moisture permeable and water resistant film supporting struts 6a are attached on the internal surface of the outer shell 4, extending from the internal surface to a direction of the center of the circle. In the present embodiment, the wire heater 8 is provided in the water retention region 10, and specifically, the wire heater 8 is attached to the moisture permeable and water resistant film supporting struts 6a. It should be noted that it is not limited thereto, and it is also possible to, for example, dispose a wire heater outside the outer shell 4 as in the first embodiment and also to load a plate heater outside the outer shell 4 as in the second embodiment.

In the present embodiment, the moisture permeable and water resistant film 6 has a wavy shape like the folds of a nasal cavity, so that the area to heat and humidify inside the aeration region 12 can be increased drastically. This enables to achieve heating and humidification of the inspiratory gas sufficient for a user more securely in the present embodiment.

An artificial nose having no heat and moisture exchanger element 14 in the aeration region 12 can be considered, and even in this case, the moisture permeable and water resistant film 6 has a wavy shape like the folds of a nasal cavity, so that the inspiratory gas passing through the aeration region 12 can be expected to be heated and humidified sufficiently.

### Description of One Embodiment of Breathing Circuit Provided with Artificial Nose According to the Invention

Then, with reference to FIG. 5, a detailed description is given to one embodiment of a breathing circuit provided with an artificial nose according to the present invention. Here, FIG. 5 is a diagram schematically illustrating each device configuring a breathing circuit 20, including the artificial nose 2.

The breathing circuit 20 of the present embodiment is provided mainly with the artificial nose 2, the Y shaped connector 36 connected to the end 2c of the inspiratory supply source of the artificial nose 2, the inspiratory tube 32 and the expiratory tube 34 connected to the bifurcated ends of the Y shaped connector 36, the inspiratory gas supply source 22 connected to the inspiratory tube 32 to supply the inspiratory gas to the inspiratory tube 32, the water supply means 30 to supply water to the water retention region 10 with an approximately constant static pressure via the feed water inlet 16, the heater output adjustment means 42, drop rate monitoring means 40, and control means 28. Although the artificial nose 2 shown in FIG. 5 is an embodiment in which the wire heater 8 wraps around the outer circumference of the outer shell 4 (refer to FIG. 2(a)), it is not limited to that and it is also possible to use an embodiment in which a plate heater is attached (refer to FIG. 3) and an embodiment in which a heater is attached in the water retention region (refer to FIG. 4(b)).

The heater output adjustment means 42 adjusts power to be supplied to the heater 8 based on a thermistor signal (temperature measurement data) mounted in the water retention region 10 of the artificial nose 2. The drop rate detection means 40 provided with the water supply means 30 measures the drop rate of the water, and the control means 28 carries out a control process of issuing a predetermined alert based on the measurement data received from the drop rate detection means 40.

By this thermistor 18 and a control device thereof, it is also possible to measure the temperature of the inspiratory gas. Therefore, in a case that the inspiratory gas exceeds a predetermined temperature (for example, 43° C.), a control process of issuing a high temperature alert can be carried out, and similarly in a case that the temperature of the inspiratory gas falls below a predetermined value due to cable disconnection of the heater or the like, a control process of issuing a low temperature alert can be carried out.

By the breathing circuit 20 with the configuration as above, the inspiratory gas supplied from the inspiratory supply source 22 is supplied to the user via the inspiratory tube 32 and the Y shaped connector 36 passing through the artificial nose 2. Meanwhile, the expiratory gas exhaled from the user passes through the artificial nose 2, via the Y shaped connector 36 and the expiratory tube 34 to be discharged to the atmosphere.

At this time, the first heating and humidifying process of the inspiratory gas is carried out in which the heat and the moisture included in the expiratory gas passing therethrough are captured and retained by the heat and moisture exchanger element 14 of the artificial nose 2 and are discharged to an inspiratory gas passing therethrough next, and also the second heating and humidifying process is carried out in which the water vapor generated by the heating of the heater 8 passes through the moisture permeable and water resistant film 6 from the water retention region 10 to heat and humidify the inspiratory gas passing through the heat and moisture exchanger element 14 of the aeration region 12 and also the inspiratory gas passing through the heat and moisture exchanger element 14 in the aeration region 12 is heated by the heater 8. A description is given below to each component device configuring the breathing circuit 20.

### <Description of Heater Output Adjustment Means 42>

The heater output adjustment means 42 of the present embodiment carries out a control process of adjusting power to be supplied to the heater 8 based on a thermistor signal (temperature measurement data) sent from the thermistor 18 mounted in the artificial nose 2. In the present embodiment, the power application to the heater 8 is controlled to keep the temperature in a region where the thermistor 18 is mounted (for example, in the water retention region 10) at 40° C. This enables to realize optimal heating and humidification of the inspiratory gas. It should be noted that the temperature settings are not limited to 40° C. and any temperature settings are possible in accordance with the usages, position settings, or the like.

Although the present embodiment is provided with the heater output adjustment means 42 that is small in size and dedicated for the artificial nose 2, it is not limited thereto and the power application to the heater 8 can also be controlled using the control means of the entire breathing circuit 20.

### <Description of Water Supply Means 30>

The water supply means 30 is provided with the water container 24 and a dropping chamber 26 having an upper portion in communication with the water container 24 and a lower portion in communication with the water supply tube 38. The upper portion of the dropping chamber 26 is provided with a pipe 26a in communication with the water container 24 and the water in the water container 24 is dropped from this pipe 26a and thus the water can be supplied to the water supply tube 38 connected to the water retention region 10 of the artificial nose 2. As already described using FIGS. 2(a) and 2(b), the water supplied to the water supply tube 38 is supplied to the water retention region 10 through the feed water inlet 16.

Firstly, a procedure of filling water in the water retention region 10 is described. As the water container 24 is attached, the water flows from the water container 24 into the water retention region 10 due to the water pressure. At this time, the air retained in the water retention region 10 permeates the moisture permeable and water resistant film 6 and escapes to the aeration region 12 side. As the inside of the water retention region 10 is filled with water, water does not flow out of the water container 24. After that, an amount of water corresponding to the amount of water vapor passed through the moisture permeable and water resistant film 6 and come out to the aeration region 12 is dropped from the pipe 26a to be supplied to the water retention region 10.

On the contrary, although there is a possibility that the inspiratory gas permeates the moisture permeable and water resistant film 6 from the aeration region 12 side to enter into the water retention region 10, the maximum pressure in artificial respiration is 100 cm H₂O or less, so that a back flow of the gas does not occur as long as the water container 24 is positioned 100 cm or more above the breathing circuit (artificial nose 2) (in FIG. 5, H>=100 cm).

For the water supply tube 38 from the water container 24 to the artificial airway 2, it is preferred to use, for example, a thin tube like one used for transfusion. Increasing the flow resistance in the tube using a thin tube enables to prevent a back flow of a gas even more effectively.

To describe the dropping chamber 26 further in detail, due to the dropping of water from the pipe 26a, water is retained in the lower portion of the dropping chamber 26 to form a water surface at a predetermined level (level shown with H). Here, the level of the water surface formed in the dropping chamber 26 is arranged so as to be higher by the difference H in height relative to the artificial airway 2.

Suppose if the level of the water surface rises in the dropping chamber 26, the air pressure in the dropping chamber 26 rises and acts to decrease the hydrostatic pressure to be a factor for water droplet formation, so that the drop rate becomes late. In contrast, suppose if the level of the water surface falls in the dropping chamber 26, the air pressure in the dropping chamber 26 falls and acts to increase the hydrostatic pressure to be a factor for water droplet formation, so that the drop rate becomes fast. Accordingly, the dropping chamber 26 has a self-adjusting function that adjusts the drop rate so as to always make the level of the water surface constant.

As described above, the level fluctuation of the water surface in the dropping chamber 26 is extremely small compared to the difference H in height with the artificial nose 2 and there is also the flow resistance of the water supply tube 38, so that the water supply means 30 can supply water to the water retention region 10 of the artificial nose 2 at a basically constant static pressure (head of water H). This enables the water retention region 10 to be supplement with water by the water supply means 30 in the amount of water corresponding to the amount of water vapor that has become water vapor by being heated by the heater 8 in the water retention region 10 of the artificial nose 2 and passed through the moisture permeable and water resistant film 6 to come out to the aeration region 12.

As described above, by applying an approximately constant static pressure (head of water H), the water retention region 10 can be supplemented with water in the amount of water corresponding to the amount of water vapor passing through the moisture permeable and water resistant film 6 and gone out, so that it becomes possible to provide the breathing circuit 20 capable of humidifying the inspiratory gas stably for a long period of time without an excessive control process.

### <Description of Drop Rate Measurement Means 40>

Then, a description is given to the drop rate measurement means 40 provided in the water supply means 30. The drop rate measurement means 40 is mounted on a side portion of the dropping chamber 26 and is arranged to drop a water droplet between a light emitting device 40a emitting a visible light at a predetermined wavelength and a light receiving device 40b. When a water droplet drops, a light incident to the light receiving device 40b from the light emitting device 40a (refer to an arrow in FIG. 5) is interrupted, so that the dropping of water can be sensed. Since a time interval between the drops can be measured by a timer built in the drop rate measurement means 40, it is possible to accurately measure the drop rate. Then, the data of the drop rate of water measured by the drop rate measurement means 40 is sent to the control means 28.

In the present embodiment, although the drop rate measurement means 40 using a visible light sensor is shown as an example, it is not limited thereto and drop rate measurement means using any other sensor, including an infrared sensor, is applicable.

### <Description of Control Means 28>

As the control means 28 of the present embodiment, a commercially available computer can also be used that is provided with a processor (CPU), memory devices (ROM and RAM), an external interface, a driving circuit, or the like.

<<Control over Drop Rate>>

The control means 28 carries out a control process of issuing a predetermined alert when the drop rate of water exceeds a predetermined value or when the drop rate falls below a predetermined value based on the drop rate measurement data sent from the drop rate measurement means 40. That is, as the amount of water flowing into the water retention region 10 of the artificial nose 2 increases for some reason, the level of the water surface of the dropping chamber 26 drops, and the drop rate rises due to the self-adjusting function included in the dropping chamber 26. On the contrary, as the amount of water flowing into the water retention region 10 of the artificial nose 2 decreases for some reason, the level of the water surface of the dropping chamber 26 rises, and the drop rate drops due to the self-adjusting function included in the dropping chamber 26. Also in a case that the water in the water container 24 becomes less, the drop rate in the dropping chamber 26 drops as well. In a case that this drop rate exceeds a predetermined value or a case that the drop rate falls below a predetermined value, a control process of issuing a predetermined alert is carried out by, for example, sounding an alarm, activating an indication lamp, or sending a signal to a hospital system.

Here, in a case that the drop rate exceeds a predetermined value, there is a high possibility that the moisture permeable and water resistant film 6 of the artificial nose 2 is damaged and the water in the water retention region 10 is leaked to the aeration region 12 side, so that promptly issuing an alert enables to prevent a user from drowning (choked by water entering into a trachea or a lung) before it happens to secure the safety of the user.

Also when the drop rate from the container containing water falls below a predetermined value, a control process of issuing an alert is carried out, so that even if the water supply tank becomes empty or water becomes not supplied to the water retention region 10 for an obstruction of the tube or the like, it is possible to issue an alert promptly to secure safety of the user.

<Description of other Control Process>

In the present invention, by, for example, temperature measurement means provided in the inspiratory tube 32, it is also possible to measure the temperature of the inspiratory gas flowing in the aeration region 12 of the artificial nose 2 and, based on the measurement data, to adjust the output of the heater provided in the inspiratory tube 32 by the control means 28 to control the inspiratory temperature. In addition, by flow rate measurement means provided in the inspiratory tube 32, it is also possible to measure the flow rate of the inspiratory gas and, based on the measurement data, to adjust the output of the inspiratory supply source 22 to control the flow rate of the inspiratory gas.

As described above, in the artificial nose 2 of the present invention and the breathing circuit 20 provided with the artificial nose 2, the second heating and humidifying process that supplies heat and moisture to the inspiratory gas by the water vapor permeated from the water retention region 10, and at the same time, supplies further heat to the inspiratory gas from the heater enables to make up for the heating and humidification of the inspiratory gas insufficient only by the first heating and humidifying process with the heat and moisture exchanger element 14 to realize heating and humidification of the inspiratory gas sufficient for a user. Further, in this embodiment, only the water vapor generated by the heating of the wire heater 8 passes through the moisture permeable and water resistant film 6, so that there is no possibility of obstructing the flow channel of the inspiratory gas and the expiratory gas by supplying excessive moisture to the heat and moisture exchanger element 14 and there is no risks of flowing excessive moisture into a trachea or a lung of a user, so that it is possible to realize humidification and heating sufficient for a user in a state of securing safety.

Further, the artificial nose 2 is warmed by a heat source of the heater along an outer circumference of the artificial nose 2, so that the artificial nose 2 itself can be less affected by the external temperature (influence due to the room temperature and an air from an air conditioner or the like) or the like to maintain stable heating and humidification.

### Description of Another Embodiment of Artificial Nose According to the Invention and Breathing Circuit Provided with the Artificial Nose

### <Description of Another Embodiment (1) of Artificial Nose According to the Invention>

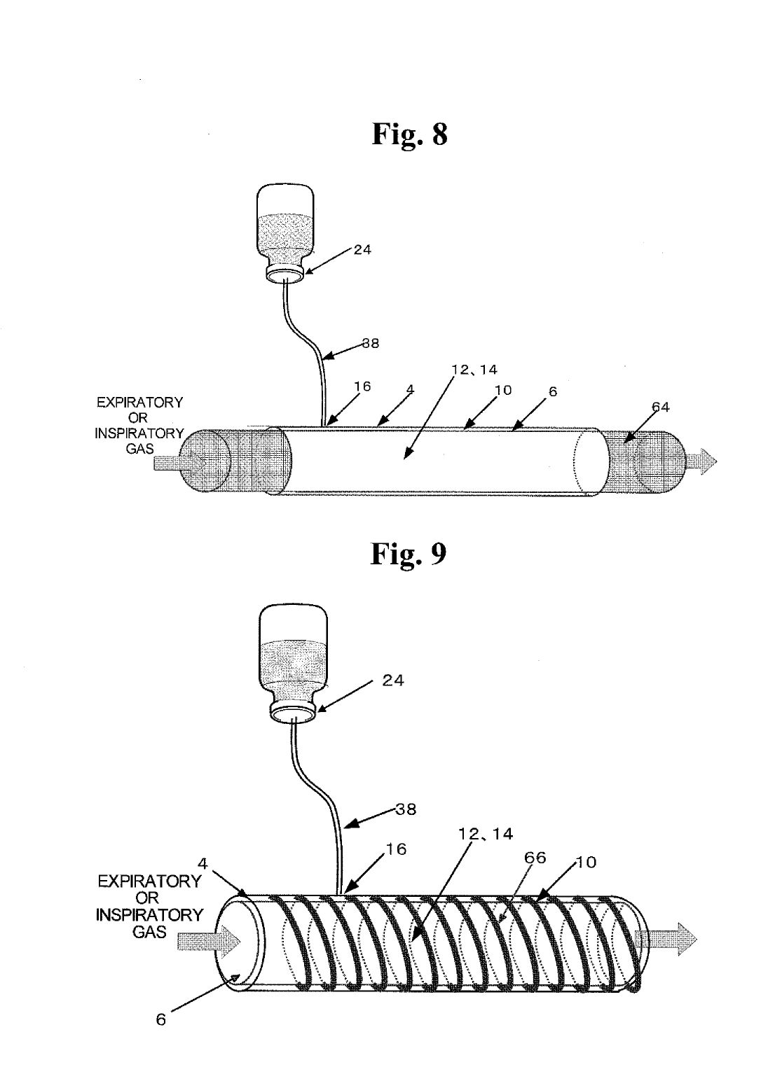

As another embodiment (1) of an artificial nose according to the present invention, a description is given to an artificial nose having a tubular reinforcement member disposed on an internal surface side of a moisture permeable and water resistant film using FIG. 8.

In FIG. 8, the artificial nose 2 is provided with the tubular outer shell 4 having air tightness and water tightness and the moisture permeable and water resistant film 6 disposed on the entire circumference of the internal surface of the outer shell 4, and further, a column net tube 64 made of a resin, which is a tubular reinforcement member, is disposed on the internal surface side of the moisture permeable and water resistant film 6 so as to make contact with the internal surface of the moisture permeable and water resistant film 6. With such a structure, the water retention region 10 is formed between the internal surface of the outer shell 4 and the outer surface of the moisture permeable and water resistant film 6, and the aeration region 12 (the heat and moisture exchanger element 14 is filled inside) is formed on the internal surface side of the moisture permeable and water resistant film 6 supported by the column net tube 64 made of a resin. The water stored in the water container 24 is led into the water retention region 10 from the feed water inlet 16 through the water supply tube 38.

In the present embodiment, the resin column net tube is used as a tubular reinforcement member 64, using a resin material and being in a mesh shape, so that it is possible to realize a reinforcement member 64 of a light weight while having sufficient strength for actual use.

It should be noted that the tubular reinforcement member 64 is not limited to those made of a resin and can use any other material, including a metal, and the shape is also not limited to a cylindrical shape and can employ any other shape and also does not necessarily have a mesh.

According to the present embodiment, even in a case that the tube configured with the moisture permeable and water resistant film 6 does not have the strength for maintaining the cylindrical shape, the column net tube 64 made of a resin (tubular reinforcement member) is disposed so as to make contact with the internal surface of the moisture permeable and water resistant film 6, so that the tube configured with the moisture permeable and water resistant film 6 can be maintained in a cylindrical shape and the moisture permeable and water resistant film can be prevented from expanding inward to secure a sufficient size of the aeration region 12 (the heat and moisture exchanger element 14 is filled inside).

### <Description of Another Embodiment (2) of Artificial Nose According to the Invention

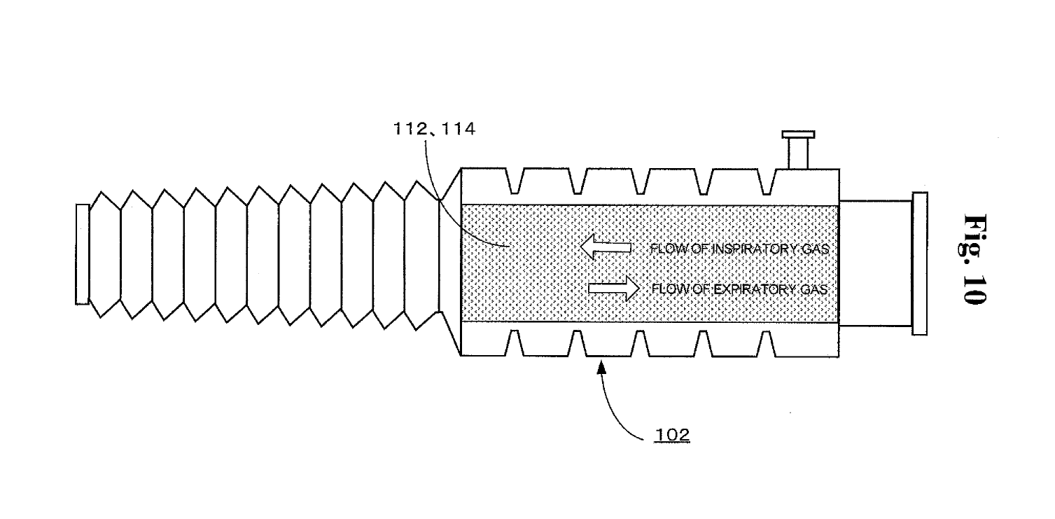

As another embodiment (2) of an artificial airway according to the present invention, a description is given to an artificial nose having a helical core disposed in the water retention region between an outer shell and a moisture permeable and water resistant film using FIG. 9.

In FIG. 8, the artificial nose 2 is provided with the tubular outer shell 4 having air tightness and water tightness and the moisture permeable and water resistant film 6 disposed on the entire circumference of the internal surface of the outer shell 4, and thus, the water retention region 10 is formed between the internal surface of the outer shell 4 and the outer surface of the moisture permeable and water resistant film 6, and the aeration region 12 (the heat and moisture exchanger element 14 is filled inside) is formed on the internal surface side of the moisture permeable and water resistant film 6. In the present embodiment, further, a helical core 66 made of a resin is disposed in the water retention region 10 between the outer shell 4 and the moisture permeable and water resistant film 6.

The water stored in the water container 24 is led into the water retention region 10 from the feed water inlet 16 through the water supply tube 38. At this time, a helical flow channel guided by the helical core 56 is formed in the water retention region 10, and the water supplied from the feed water inlet 16 can stream entirely in the water retention region 10 along this helical flow channel.

Although the helical core 66 of the present embodiment is made of a resin, it is not limited to that and any other material, including a metal, can be used, and the shape is also not limited to a cylindrical shape and any other shape can be employed.

To form this artificial nose 2, it can be realized by, for example, adhering the moisture permeable and water resistant film 6 to inside the helical core 66 and adhering the outer shell 4 to outside the helical core 56, and seal bonding the moisture permeable and water resistant film 6 and the outer shell 4 at both ends.

According to the present embodiment, even in a case that the tube configured with the moisture permeable and water resistant film 6 does not have the strength for maintaining the cylindrical shape, the helical core 66 is disposed in the water retention region 10, so that the tube configured with the moisture permeable and water resistant film 6 can be maintained in a cylindrical shape and the moisture permeable and water resistant film 6 can be prevented from expanding inward to secure a sufficient size of the aeration region 12 (the heat and moisture exchanger element 14 is filled inside). In addition, the water flows along the helical flow channel formed with the helical core 66, so that the helical core 66 does not impede the flow of water in the water retention region 10.

Regarding the aspect ratio (ratio of the diameter of the cross-section and the longitudinal length) of the tubular outer shell 4, the resin column net tube 64, the helical core 66, or the like shown in FIGS. 8 and 9, those having any profile can be employed including not only the profiles as shown in FIGS. 8 and 9 but also those shown in FIGS. 1 through 5.

### <Description of Another Embodiment (3) of Artificial Nose According to the Invention>

An artificial nose according to the present invention may also not be used for a breathing circuit. For example, in a case of making a patient in whom a tracheostomy is performed to breathe spontaneously in a state of being loaded with a trachea tube, an artificial nose is attached at the tip end of the intratracheal tube for spontaneous breathing from the atmosphere. This enables the artificial nose to substitute the upper trachea to some extent even when the upper trachea (area from the nose to the throat) is bypassed.

Embodiments of an artificial nose according to the present invention and a breathing circuit provided with the artificial nose are not limited to the above embodiments, and the present invention includes any other embodiments.

1. An artificial nose, comprising:
an outer shell;
a moisture permeable and water resistant film disposed on an entire circumference of an internal surface of the outer shell, forming a water retention region with the outer shell, and forming an aeration region on an internal surface side thereof;
a feed water inlet provided in the outer shell to supply water to the water retention region;
a heat and moisture exchanger element loaded in the aeration region; and
a heater disposed outside the outer shell,
wherein the water supplied from the feed water inlet is retained in the water retention region by the moisture permeable and water resistant film,
an inspiratory gas and an expiratory gas pass through the heat and moisture exchanger element loaded in the aeration region, and
the artificial nose carries out a first heating and humidifying process of the inspiratory gas in which heat and moisture included in the expiratory gas passing therethrough are captured and retained by the heat and moisture exchanger element and the heat and the moisture are discharged to an inspiratory gas passing therethrough next, and
a second heating and humidifying process in which only water vapor generated by heating of the heater passes through the moisture permeable and water resistant film and is supplied to the inspiratory gas passing through the heat and moisture exchanger element to heat and humidify the inspiratory gas and also the inspiratory gas in the heat and moisture exchanger element is heated by the heater.

2. The artificial nose according to claim 1, wherein the heater is configured with a wire heater that wraps around outside the outer shell in a region where the water retention region is formed.

3. The artificial nose according to claim 1, wherein the heater is configured with a plate heater disposed outside the outer shell in a region where the water retention region is formed.

4. The artificial nose according to claim 1, wherein the heating and humidification of the inspiratory gas is possible to be adjusted at the same time by adjusting a power application to the heater.

5. The artificial nose used for a breathing circuit according to claim 1, wherein the moisture permeable and water resistant film includes a resin made sheet or a resin made film.

6. The artificial nose used for a breathing circuit according to claim 1, wherein the moisture permeable and water resistant film includes a nonwoven fabric having moisture permeability and water resistance.

7. The artificial nose used for a breathing circuit according to claim 1, wherein the moisture permeable and water resistant film includes a porous material or a nonporous material.

8. The artificial nose used for a breathing circuit according to claim 1, wherein the heat and moisture exchanger element is configured with a resin made foam, a resin fiber tangled like cotton wool, or hygroscopic paper.

9. The artificial nose used for a breathing circuit used for a breathing circuit according to claim 1, wherein a tubular reinforcement member is disposed on the internal surface side of the moisture permeable and water resistant film to make contact with the internal surface.

10. The artificial nose used for a breathing circuit according to claim 1, wherein a helical core is disposed in the water retention region between the outer shell and the moisture permeable and water resistant film and the water supplied from the feed water inlet flows along a helical flow channel formed with the helical core.

11. A breathing circuit, comprising:
the artificial nose according to claim 1;
an inspiratory tube and an expiratory tube in communication with one end of the aeration region of the artificial nose;
an inspiratory gas supply source supplying the inspiratory gas to the inspiratory tube; and
water supply means supplying the water to the water retention region with an approximately constant static pressure via the feed water inlet, wherein
the water retention region is supplemented with water by the water supply means in an amount of water corresponding to an amount of water vapor passed through the moisture permeable and water resistant film and flown out.

12. The breathing circuit according to claim 11, wherein the water supply means supplies the water by dropping from a container that contains the water and includes:
drop rate measurement means measuring a rate of the dropping; and
control means carrying out a control process of issuing an alert, based on drop rate measurement data sent from the drop rate measurement means, when the drop rate exceeds a predetermined value or when the drop rate falls below a predetermined value.
